(19) United States
(12) Patent Application Publication
(10) Pub. No.: US 2025/0127936 A1
(43) Pub. Date: Apr. 24, 2025

(54) Contrast agent for magnetic resonance imaging

(71) Applicant: Betavist GmbH, Pfäffikon (CH)

(72) Inventor: Yelda Özsunar, Aydin (TR)

(73) Assignee: Betavist GmbH, Pfäffikon (CH)

(21) Appl. No.: 18/570,180

(22) PCT Filed: Jun. 20, 2022
(86) PCT No.: PCT/EP2022/066734

§ 371 Date: Dec. 14, 2023

(30) Foreign Application Priority Data
Jun. 22, 2021 (TR) 2021/010176
Jul. 8, 2021 (WO) PCT/TR2021/050696

(51) Int. Cl. A61K 49/10; A61K 36/21

(52) U.S. Cl. CPC A61K 49/10; A61K 36/21; A61K 2236/15; A61K 2236/331; A61K 2236/37; A61K 2236/53

(57) ABSTRACT

The present invention relates to a contrast agent composition for magnetic resonance imaging. The contrast agent composition contains water, and one or more chemically distinct betalains. The contrast agent composition is suitable for oral, rectal and/or parenteral administration. In one embodiment, the composition is a Beta vulgaris juice.

### FIELD OF THE INVENTION

The present invention relates to the technical field of contrast agents for magnetic resonance imaging.

### BACKGROUND OF THE INVENTION

A contrast agent is a chemical substance given to the body from outside in order to increase the contrast of the tissues and liquids in the body during medical imaging. Contrast agents are commonly used in imaging such as Fluoroscopy, Computerized Tomography and Magnetic Resonance to increase the visibility of blood vessels and organs (stomach, intestines etc.), tumors, and inflammatory tissues. The contrast agents used in medical imaging can be roughly classified according to the imaging methods in which they are used.

Magnetic resonance imaging (MRI) is a method for acquiring anatomical, physiological, and biochemical information on the body as images using a phenomenon in which the spins of hydrogen atoms are relaxed in a magnetic field. MRI is one of the current noninvasive diagnostic tools for real-time imaging of the body organs of living humans and animals. For its diverse and precise use in the bioscience and medical fields, MRI is performed by introducing foreign materials into the body to increase the contrast of images. These materials are called contrast agents. Superparamagnetic and paramagnetic materials are used as contrast agents to contrast signals from body parts to be imaged by MRI so that the body parts can be clearly distinguished from their surroundings. Contrast between tissues on an MRI image arises because of different relaxations in the tissues. The relaxation is a phenomenon in which the nuclear spins of water molecules in the tissues return to their equilibrium state. A contrast agent affects the relaxations to create large differences in the degree of relaxation between the tissues and induces changes of MRI signals to make the contrast between the tissues clearer.

Enhanced contrast using contrast agents raises or lowers the intensities of image signals from specific living organs and tissues relative to their surroundings to provide clearer imaging of the organs and tissues. Positive contrast agents (or T1 contrast agents) refer to contrast agents that raise the intensities of image signals from body parts to be imaged by MRI relative to their surroundings. Negative contrast agents (or T2 contrast agents) refer to contrast agents that lower the intensities of image signals from body parts to be imaged by MRI relative to their surroundings. More specifically, MRI contrast agents are divided into T1 contrast agents using high spins of paramagnetic materials and T2 contrast agents using magnetic inhomogeneity around ferromagnetic or superparamagnetic materials.

Intravenous gadolinium (Gd) chelates, such as gadobutrol, and gadotheric acid, are currently used as T1 contrast agent for MRI. As well known, in many situations that need visualization of tumors, infections and vessels in unenhanced imaging where tissue distinguishability cannot be sufficiently achieved, gadolinium chelate paramagnetic products are used where considered necessary for lesion and diagnostic evaluations. These gadolinium derivatives are being commonly used for diagnosis and follow-up of diseases. Approximately 30 million doses of gadolinium chelate are used per year for diagnosis purposes. While gadolinium containing contrast agents can be intaken in chelate forms, they become toxic both for the patient and for the environment if the Gd³⁺ cation is separated from its ligand(s). The gadolinium containing contrast agents were considered safe until 2006, when it was reported that such products cause side effects, such as nephrogenic systemic fibrosis. The number of publications reporting on the side effects of gadolinium containing contrast agents has significantly increased since 2013. Ligand free gadolinium both in its free form in the body and blended into natural environment by urine poses risk in terms of environmental health and ecological balance. Intravenous (iv) injection of these products increases the risk of systemic side effects in patients with diabetes, hypertension, liver failure and especially kidney function failure. In addition, it has been shown that gadolinium chelates cause accumulation in skin, liver, bone and brain (Guo et al. Frontiers in Molecular Neuroscience, 2018, 11, article 335). The oral, anal, and rectal administration of these products is unpractical, inconvenient and associated with dilution difficulties. Although gadolinium-based contrast agents could be administered orally, they are not routinely used because difficulty of administration requiring dilution and the poor quality of the obtained MRI images. Furthermore, gadolinium-based contrast agents cannot be administered anally and rectally because of their low viscosity.

Regardless of the side effects and risks associated with their administration, gadolinium chelates remain the main contrast agent currently used for MRI.

Hence, a need remains for a harmless contrast agent for magnetic resonance imaging, in particular for stomach and intestinal examinations.

### SUMMARY OF THE INVENTION

Accordingly, it is an object of the present invention to provide a contrast agent composition for magnetic resonance imaging, wherein said composition contains water, and one or more chemically distinct betalains.

Also claimed and described herein is a process for manufacturing the contrast agent composition described herein, wherein said process contains the following steps:
a) peeling and slicing a Beta vulgaris plant, preferably a Beta vulgaris plant originating from the Aydin region (Turkey);
b) pulping the Beta vulgaris plant to provide a pulp;
c) mixing the pulp obtained at step b) with water;
d) boiling the mixture obtained at step c);
e) following cooling, draining the mixture to separate a liquid; and
f) adding an aqueous solution containing a betanin and/or a vulgaxanthin to the liquid obtained at step e), as well as a contrast agent composition obtained by said process. When added together, the weight ratio of betanin to vulgaxanthin is preferably of 3:1.

A further aspect according to the present invention relates to a method of acquiring a contrast enhanced magnetic resonance image of a subject, said method comprising
administering to said subject the contrast agent composition described herein; and
acquiring said magnetic resonance image of said subject.

### DESCRIPTION OF THE FIGURES

FIG. 1 shows an overview of a manufacturing process of a contrast agent composition according to the present invention: (10) washing with water; (20): slicing; (30): pulping; (40): boiling; (50): cooling; (60): draining; (70): separating the liquid part; (80): adding flavor enhancers.

FIG. 2 shows an overview of a manufacturing process of a contrast agent composition according to the present invention: (10) washing with water; (20): slicing; (30): pulping; (40): boiling; (50): cooling; (60): draining; (70): separating the liquid part; (80): adding flavor enhancers; (90): obtaining the extract; (100): adding into the mix.

FIG. 3 shows a comparative schematic view of the T1 relaxation curves measured for the contrast agent compositions CA1 and CA2 according to the present invention and the comparative gadolinium chelate composition (C1) (x axis: time of repetition (TR)/ms; y axis: T1 average image signal intensity over selected region of interest. The contrast compositions CA1 and CA2 according to the present invention exhibit a spin echo T1 comparable with the currently used gadolinium chelate composition C1. Hence, contrast compositions CA1 and CA2 are suitable to be used as a contrast agent for in vivo magnetic resonance imaging.

FIG. 4 compares the magnetic resonance contrast between spin echo T1 weighted images obtained in vitro with the contrast agent composition CA3 according to the present invention (top left), the comparative gadolinium chelate solution C1 (top right), water as reference (bottom left), and olive oil (bottom right), respectively, and in vivo on a rat under anesthesia (middle). As shown by FIG. 4, the contrast agent composition CA3 shows a significantly higher signal intensity than the comparative gadolinium chelate composition C1, water, olive oil and the rat tissue under anesthesia.

FIG. 5 shows spin echo T1 weighted images obtained on a rat following rectal administration of the contrast agent composition CA3 (images a and b), given together with images showing the rat intestine anatomy (images c and d). As shown by images a and b, the contrast agent composition CA3 shows bright MRI contrast in intestinal lumen following rectal administration and enables better delineation of the bowel lumen. Hence, the contrast agent composition CA3 is particularly suitable for lower gastrointestinal imaging.

FIG. 6 shows sequential spin echo T1 weighted images obtained on a rat following oral administration of the contrast agent composition CA2. As illustrated by FIG. 6, the contrast agent composition CA2 shows bright MRI contrast in stomach following oral administration and enables better delineation of the stomach lumen. Hence, the contrast agent composition CA2 is particularly suitable for gastro-esophageal imaging. The black portions noticed in the stomach correspond to sawdust.

FIG. 7 A) shows early phase (acquisition after first minute post administration) spin echo T1 weighted image (image a), early phase (acquisition after first minute post administration) fat saturated T2 weighted imaged (image b), and early phase (acquisition after first minute post administration) sequential gradient echo T1 weighted images (images c and d), and late phase (acquisition after three hours post administration) spin echo T1 weighted image (image e) obtained on a rat after oral administration of the contrast agent composition CA2 according to the present invention. As can be seen from FIG. 7 A), contrast agent composition CA2 enables delineation of the small intestine at early phase and of the liver and kidneys at late phase post oral administration and is particularly useful for upper gastrointestinal imaging at early, and parenchymal imaging at late phase.

B) shows early phase (acquisition after first minute post administration) spin echo T1 weighted images (image a and b), and early phase (acquisition after first minute post administration) sequential gradient echo T1 weighted images (image c) obtained on a rat after oral administration of the comparative gadolinium chelate composition (C1).

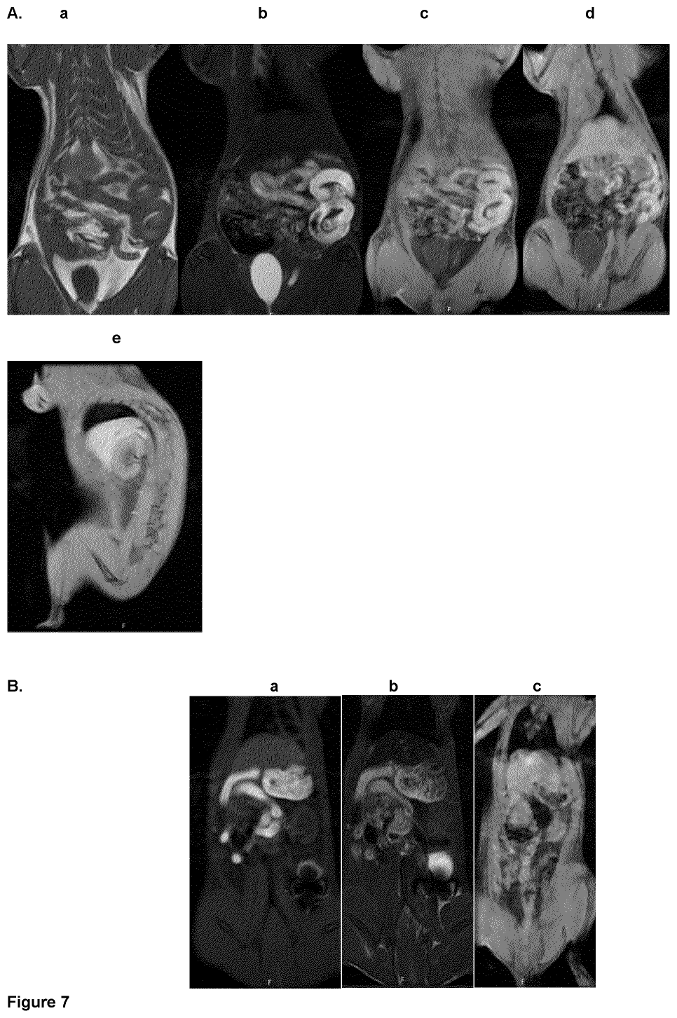

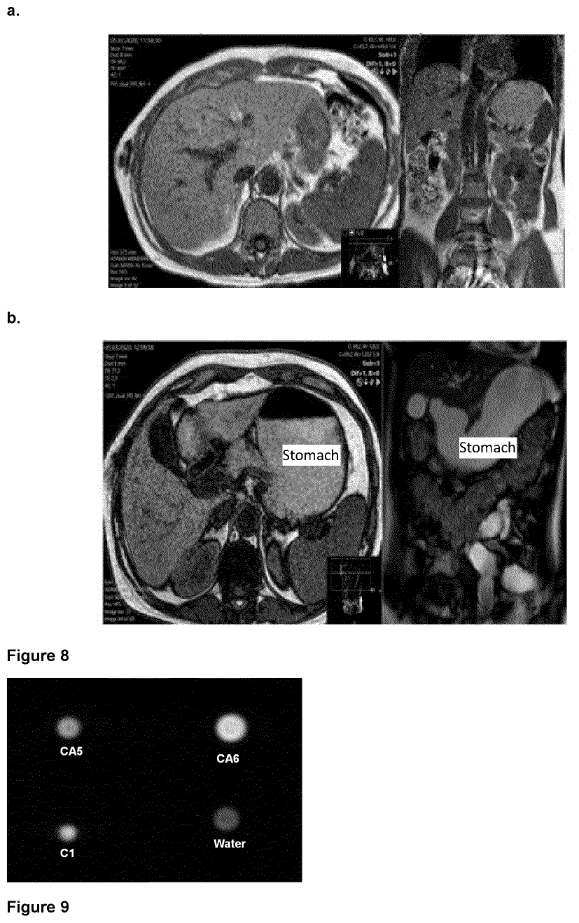

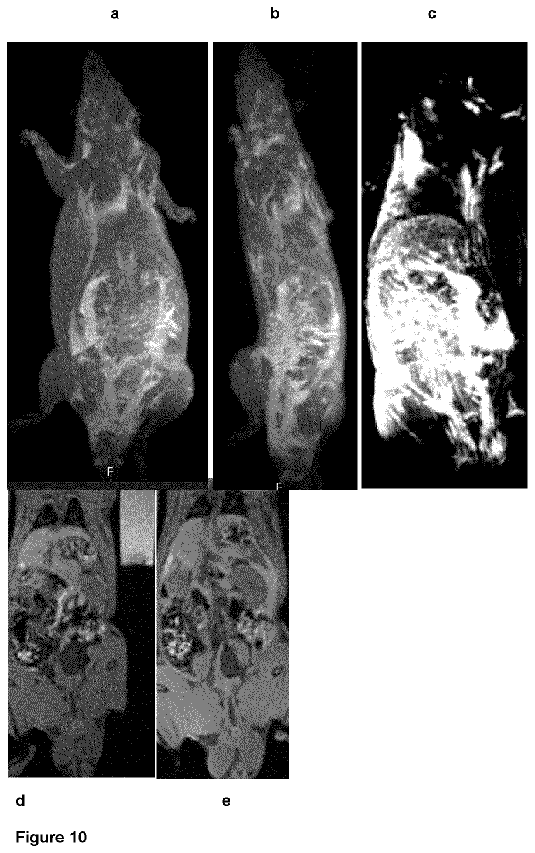

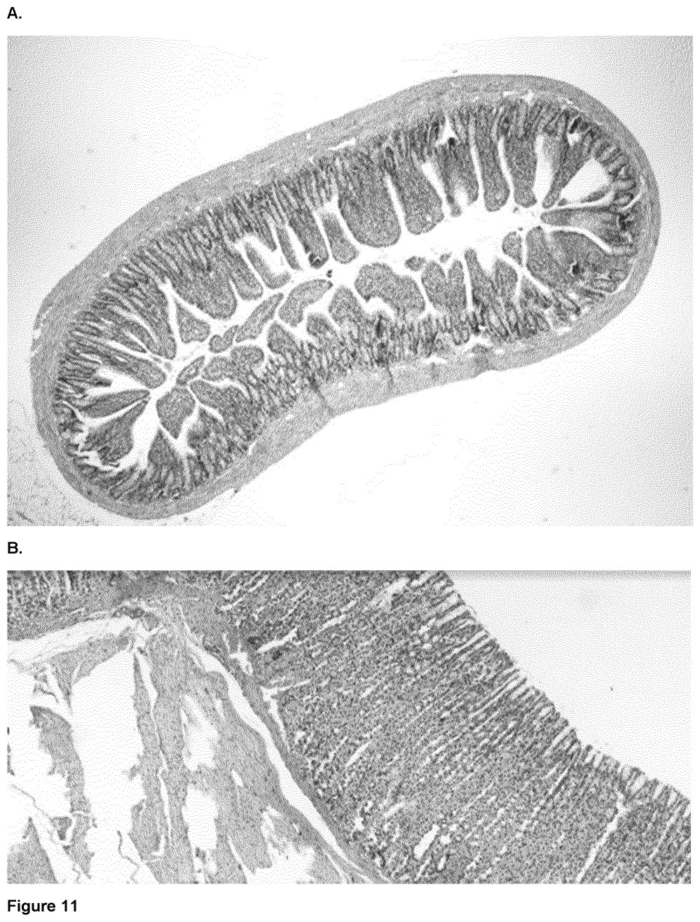

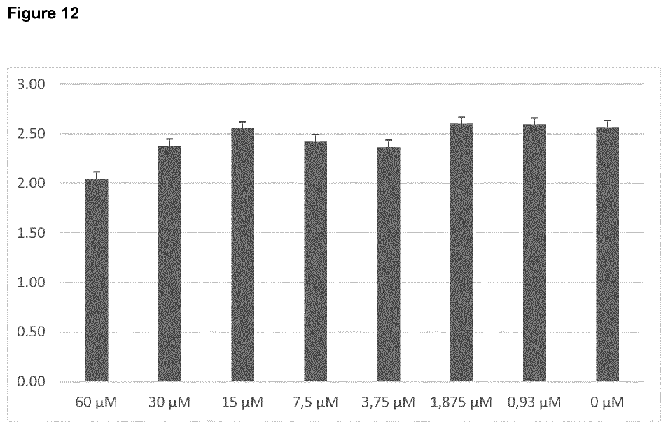

Comparison of the results depicted in FIGS. 7A and 7B demonstrates that the contrast composition according to the present invention shows better distention and delineation of the lumen of small bowel specially at sequential gradient echo T1 weighted imaging than the currently used gadolinium chelate composition (C1). This is particularly advantageous because the measurement of a gradient echo T1 weighted image is more expedient than the one of a spin echo T1 weighted image as shown on a LAVA ASPIR MRI sequence.

FIG. 8 shows in vivo MRI images of the abdomen of the inventor prior (FIG. 8 a: left image: axial in-phase gradient echo (GRE) T1 weighted image (WI); right image: coronal in phase GRE T1 WI) and after oral administration of a freshly pressed beetroot juice (FIG. 8 b: left image: axial out-of-phase GRE T1 WI right image: coronal GRE T2 WI). As show by FIG. 8, the freshly pressed freshly pressed beetroot juice enables better delineation of human stomach lumen and wall following oral administration and is particularly useful for gastric imaging.

FIG. 9 compares the magnetic resonance contrast between spin echo T1 weighted images obtained in vitro with the contrast agent composition CA5 according to the present invention, the contrast agent composition CA6 according to the present invention, the comparative gadolinium chelate solution C1, and water as reference. As shown by FIG. 9, the contrast agent composition CA5 shows a T1 signal intensity comparable to the comparative gadolinium chelate composition C1. Furthermore, the contrast agent composition CA6 shows a T1 signal intensity significantly higher than the comparative gadolinium chelate composition C1.

FIG. 10 shows early (acquisition after first minute post manual injection) rat substracted angiographic imaging (image a), late phase (acquisition after second minute post manual injection) rat substracted angiographic imaging (image b) and late phase (acquisition after third minute post manual injection) rat substracted abdominal imaging (image c) following manual tail injection of the contrast composition CA4 according to the present invention. FIG. 10 shows venous phase and parenchymal phase (liver) and demonstrates that the composition CA4 is particularly useful for vessels and parenchymal imaging following intravenous administration.

FIG. 11 A) shows a microscopic image of a histhological sample from the experimented rat's bowel following oral administration for four consecutive days of the contrast composition CA2 according to the present invention.

B) shows a microscopic image of a histhological sample from the experimented rat's stomach following oral administration for four consecutive days of the contrast composition CA2 according to the present invention.

The results displayed by FIG. 11 demonstrate that there is no toxic effect associated with the oral administration of a dosis of 1 mL of the contrast agent composition CA2 according to the present invention for four consecutive days.

FIG. 12 shows the cells proliferation levels in human healthy fibroblast (HFB) cells after administration of a contrast agent composition according to the present invention (CA2) at a concentration of between 0-60 μM (x-axis: applied concentrations of CA2, y-axis: absorbance values measured in the spectrophotometry device (TECAN Sunrise)): the effect of CA2 administered between 0-60 μM in human healthy fibroblast (HFB) cells on the viability of cells after 24 hours of incubation. The cell proliferation levels were determined spectrophotometrically after the applied XTT test. No statistically significant difference was observed.

### DETAILED DESCRIPTION OF THE INVENTION

It is an object of the present invention to address this need. The objective is achieved by the contrast agent composition recited by claim 1 and the contrast agent composition according to claim 11, and the method of acquiring a contrast enhanced magnetic resonance image of a subject according to claim 12. Preferred embodiments are disclosed in the specification and the dependent claims.

The present invention will be described in more detail below.

Where the present description refers to “preferred” embodiments/features, combinations of these “preferred” embodiments/features are also deemed to be disclosed as long as the specific combination of the “preferred” embodiments/features is technically meaningful.

Unless otherwise stated, the following definitions shall apply in this specification:

As used herein, the term “a”, “an”, “the” and similar terms used in the context of the present invention (especially in the context of the claims) are to be construed to cover both the singular and plural unless otherwise indicated herein or clearly contradicted by the context.

As used herein, the term “and/or” means that either all or only one of the elements of said group may be present. For example, “A and/or B” means “only A, or only B, or both A and B”. In the case of “only A”, the term also covers the possibility that B is absent, i.e. “only A, but not B”.

As used herein, the terms “including”, “containing” and “comprising” are used herein in their open-ended, non-limiting sense. It is understood that the various embodiments, preferences and ranges may be combined at will. Thus, for instance a solution comprising a compound A may include other compounds besides A. However, the term “comprising” also covers, as a particular embodiment thereof, the more restrictive meanings of “consisting essentially of” and “consisting of, so that for instance “a solution comprising A, B and optionally C” may also (essentially) consist of A and B, or (essentially) consist of A, B and C. As used herein, the transitional phrase “consisting essentially of” (and grammatical variants) is to be interpreted as encompassing the recited materials or steps “and those that do not materially affect the basic and novel characteristic(s) of the claimed invention. Thus, the term “consisting essentially of” should not be interpreted as equivalent of “comprising”.

As used herein, the term “about” means that the amount or value in question may be the specific value designated or some other value in its neighborhood. Generally, the term “about” denoting a certain value is intended to denote a range within ±5% of the value. As one example, the phrase “about 100” denotes a range of 100±5, i.e. the range from 95 to 105. Preferably, the range denoted by the term “about” denotes a range within ±3% of the value, more preferably ±1%. Generally, when the term “about” is used, it can be expected that similar results or effects according to the invention can be obtained with in a range of ±5% of the indicated value.

Betalains are non-toxic, ecofriendly natural pigments generally isolated from beetroot (Beta vulgaris). Betalains have been largely used as colorants in food products. The antioxidant activity of betalains is known in the literature (Egypt. J. Agric. Res. 91(3), 2013). Udonkang et al. (Biomed Hub 2018, 3, 492828) described the use of aqueous and ethanol extracts of beetroot for staining basic histological tissue structures and their use as ecofriendly alternative to hematoxylin and eosin.

Surprisingly, it has been found that a composition containing
i) water, and
ii) one or more chemically distinct betalains, is useful as a contrast agent for magnetic resonance imaging. The composition described herein may be administered orally, rectally or parenterally and allows acquisition of magnetic resonance images of the esophagus, the gastro-intestinal tract, the liver, the kidney, the venous system and/or the arterial system of a subject. The MRI contrast obtained with the composition described herein is similar to or better than the MRI contrast obtained with the commercially available gadolinium chelate Gadovist®. Advantageously, the composition described herein exhibits antioxidant and anti-inflammatory properties and no toxic effect could be identified in connection with its administration.

Preferably, the one or more chemically distinct betalains are selected from compounds of general formula (I)

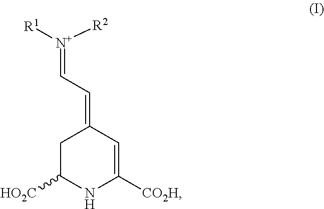

wherein in general formula (I)
either
   R¹ represents —H, and R² is selected from

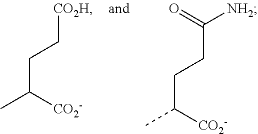

or
   R¹ and R² together with the nitrogen to which they are attached form a residue of general formula (II) or (III)

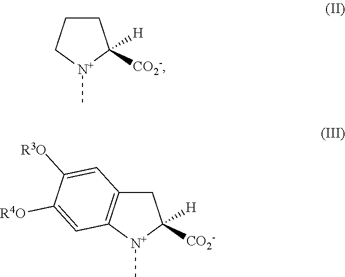

wherein in general formula (III),
   R³ and R⁴ are independently of each other selected from
      —H, and

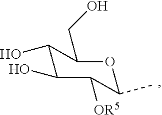

with the proviso that at least one of R³ and R⁴ represents —H, and
   R⁵ is selected from —H and

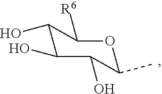

with R⁶ being selected from —CH₂OH and CO₂H, pharmaceutically acceptable salts thereof, and tautomers thereof.

The term “salt(s)” includes betalain salts that are prepared with relatively non-toxic acids or bases. Salts may be obtained by contacting the betalain with a base and/or an acid in a suitable inert solvent. Salts obtained by treatment of the betalain with a base include sodium, potassium, calcium, ammonium, and magnesium salts. Salts obtained by treatment of betalain with an acid include the salts derived from inorganic acids such as hydrochloric, hydrobromic, nitric, monohydrogencarbonic, phosphoric, monohydrogenphosphoric, dihydrogenphosphoric, and sulfuric acids, as well as the salts derived from relatively nontoxic organic acids like acetic, propionic, isobutyric, butyric, maleic, malic, and the like. The term “pharmaceutically acceptable salt” refers to those salts that are, within the scope of sound medical judgment, suitable for use in contact with the tissues of humans and lower animals without undue toxicity, irritation, allergic response and the like and are commensurate with a reasonable benefit/risk ratio.

In a preferred embodiment, the one or more chemically distinct betalains comprise a compound of general formula (IV)

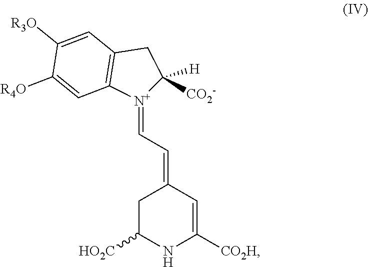

wherein R³ and R⁴ have the meanings defined herein, a pharmaceutically acceptable salt thereof and/or a tautomer thereof, and
   a compound of general formula (V)

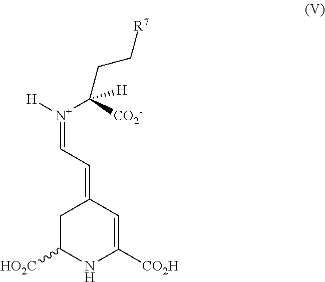

wherein in general formula (V) R⁷ is selected from —CO₂H and —CONH₂, a pharmaceutically acceptable salt thereof and/or a tautomer thereof.

In an alternative further preferred embodiment, the one or more chemically distinct betalains are selected from compounds of general formula (IV)

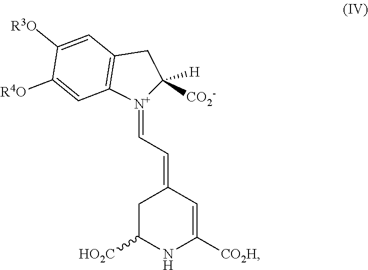

wherein R³ and R⁴ have the meanings defined herein, pharmaceutically acceptable salts thereof, and tautomers thereof.

Preferably, in general formula (IV), R⁴ represents —H. Also preferably in general formula (IV), R⁵ represents —H.

In a preferred embodiment, the compound of general formula (IV) is a betanin having the chemical structure IV-a, or a betanin having the chemical structure IV-b.

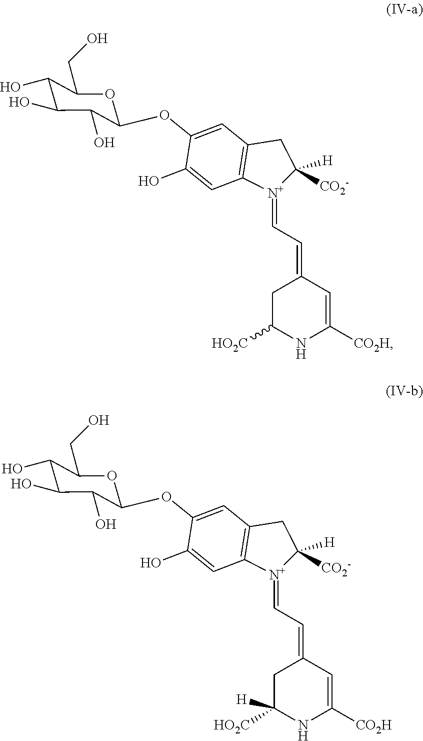

Preferably, the compound of formula (IV), (IV-a) or (IV-b) is present in the composition in an amount from about 2 g per kg of composition (2 g/kg) to about 10 g per kg of composition (10 g/kg) as determined by High Pressure Liquid Chromatography (HPLC).

Advantageously, the composition described herein is an entirely natural product. In a preferred embodiment, the composition is a Beta vulgaris juice or a beetroot juice. Preferably the juice is obtained from a beetroot originating from the Aydin region (Turkey). In another preferred embodiment, the composition is obtained by the process containing the steps:
a) peeling and slicing a Beta vulgaris plant, preferably a Beta vulgaris plant originating from the Aydin region (Turkey);
b) pulping the Beta vulgaris plant to provide a pulp;
c) mixing the pulp obtained at step b) with water;
d) boiling the mixture obtained at step c);
e) following cooling, draining the mixture to separate a liquid; and optionally
f) adding an aqueous solution containing a betanin and/or a vulgaxanthin to the liquid obtained at step e). The aqueous solution added at step f) preferably contains betanin and vulgaxanthin. When added together, the weight ratio of betanin to vulgaxanthin is preferably of 3:1.

The composition described herein may be orally administered for obtaining magnetic resonance images of the esophagus, the gastro-intestinal tract, the liver, and/or the kidney of a subject, or parenterally, preferably intravenously, or intraperitoneally, more preferably intravenously.

The parenteral administration enables acquisition of magnetic resonance images of the arterial system and/or venous system of a subject. When administered parenterally, the composition described herein may further contain an isotonic agent. Examples of isotonic agents, include but are not limited to, sodium chloride, potassium chloride, calcium chloride, sodium carbonate, dextrose, and mixtures thereof. Preferably, the isotonic agent is sodium chloride, dextrose, or a mixture of sodium chloride, potassium chloride, calcium chloride and sodium bicarbonate.

The composition for oral administration may further contain a flavoring agent to improve its palatability. Examples of flavoring agents include garlic, saffron and cilantro. If the composition is obtained with the preparation process containing step a) to e), or a) to f), the flavoring agent can be added to the liquid obtained at step e) or to the liquid obtained at step f). To increase its mucoadhesive properties, the composition may contain an adhesion enhancing substance. Such compositions are particularly useful for the acquisition of magnetic resonance images of the gastro-intestinal tract. Examples of suitable adhesion enhancing substances include, but are not limited to sucralfate, colloidal bismuth, carbenoxolone disodium salt, gelatin, pectin, sodium alginate, tragacanth, and carrageenan. The adhesion enhancing substances may be provided under microsphere form, including microcapsules and microparticles.

The composition described herein may further contain up to 50 vol-% olive oil, preferably from 10 to 50 vol-% olive oil. The olive oil may be subjected to ozonization. Such composition is particularly useful for rectal administration and enables acquisition of magnetic resonance images of the gastro-intestinal tract of a subject. Hence, a preferred embodiment according to the present invention is directed to the use of a composition containing
i) water,
ii) one or more chemically distinct betalains as described herein,
iii) up to 50 vol-%, preferably from 10 to 50 vol-%, olive oil, as a contrast agent for magnetic resonance imaging. Such composition may be obtained via a preparation process containing steps a)-e) as described herein or steps a)-f) as described herein, and additionally step g) preferably conducted after step e) or step f):
g) adding up to 50 vol-%, preferably from 10 to 50 vol-%, olive oil to the liquid obtained at step e) or f).

The composition intended for rectal administration may additionally contain an adhesion enhancing substance as described herein.

In a preferred embodiment, the composition described herein is subjected to ozonization.

A further aspect according to the present invention is directed to a process for manufacturing the contrast agent composition described herein, wherein said process contains the following steps:
a) peeling and slicing a Beta vulgaris plant, preferably a Beta vulgaris plant originating from the Aydin region (Turkey);
b) pulping the Beta vulgaris plant to provide a pulp;
c) mixing the pulp obtained at step b) with water;
d) boiling the mixture obtained at step c);
e) following cooling, draining the mixture to separate a liquid; and
f) adding an aqueous solution containing a betanin and/or a vulgaxanthin, to the liquid obtained at step e). The aqueous solution added at step f) preferably contains betanin and vulgaxanthin. When added together, the weight ratio of betanin to vulgaxanthin is preferably of 3:1.

The process may further contain a step g) conducted after step e) or f) and/or a step h):
g) adding up to 50 vol-%, preferably from 10 to 50 vol-%, olive oil to the liquid obtained at step e) or f);
h) subjecting the composition to ozonization.

A further aspect according to the present invention relates to a contrast agent composition for magnetic resonance imaging obtained by the process claimed and described herein.

The present invention further provides a method of acquiring a contrast enhanced magnetic resonance image of a subject, said method comprising
administering to said subject the composition described herein; and
acquiring said magnetic resonance image of said subject.

The actual volume of the administered composition, and the rate and time-course of administration will depend on the composition, the target area to be imagined and the subject. Prescription of the contrast agent (e.g. decision on the dosage) is within the ordinary skills of radiologists and other medical providers, and typically takes into account the disorder to be imagined and the weight of the subject.

In a preferred embodiment, the contrast agent composition is administered orally. In an alternative embodiment, the contrast agent composition is administered rectally. Rectal and oral administration of the composition according to the present invention is particularly suitable for rendering visible the esophagus, the gastro-intestinal tract, the liver, and/or the kidney of a subject via magnetic resonance imaging.

In an alternative preferred embodiment, the composition is administered parenterally, preferably intravenously, or intraperitoneally. Parenteral administration of the composition claimed and described herein enables obtaining images of the venous system and/or the arterial system of a subject.

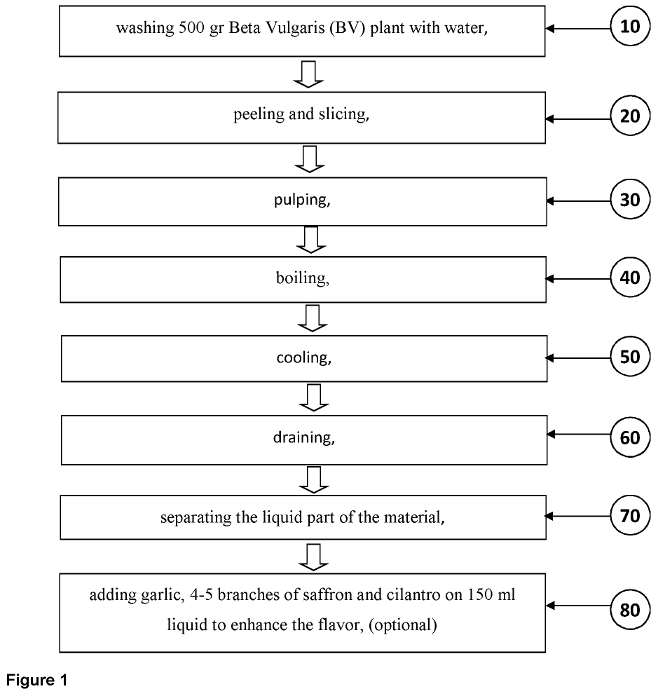

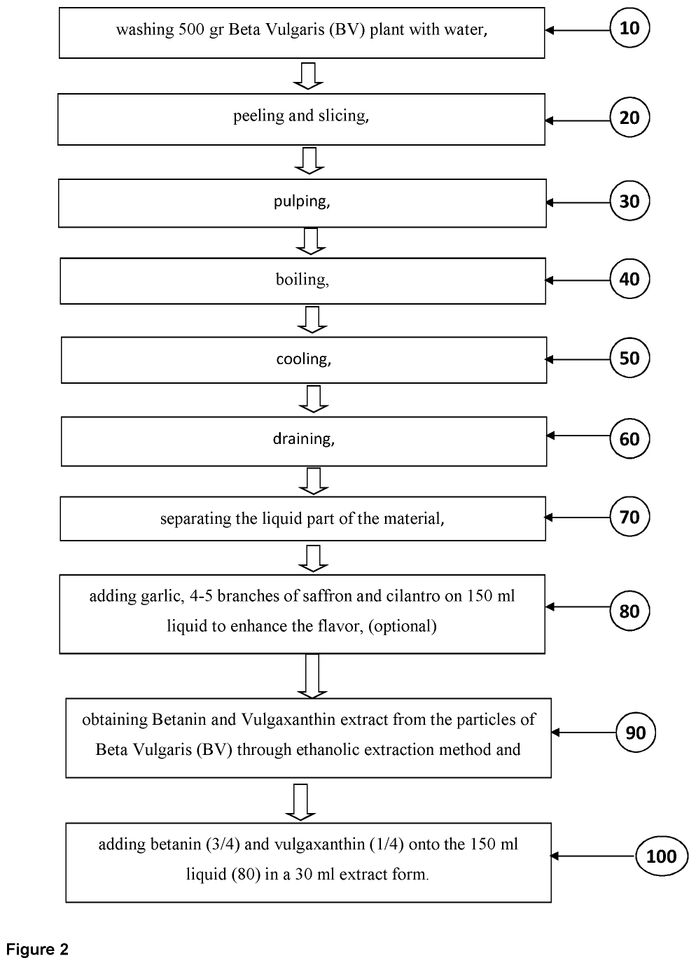

In a preferred embodiment, the contrast agent composition is obtained by the following steps of processes; washing 500 gr of Beta Vulgaris (BV) plant with water (10), peeling and slicing it (20), pulping it (30), boiling it (40), cooling it (50), draining it (60), separating the liquid part of the material (70), adding garlic, 4-5 branches of saffron and cilantro on 150 ml liquid to enhance the flavor (80), obtaining betanin and vulgaxanthin extract from the particles of Beta Vulgaris (BV) through ethanoic extraction method (90) and adding betanin (3/4) and vulgaxanthin (1/4) onto the 150 ml liquid (80) in a 30 ml extract form (100). (FIG. 1, FIG. 2). If the purpose is to use it rectally and view the upper gastrointestinal system, ozonized olive oil can be added onto it at a ratio of %10-50. (FIG. 1, FIG. 2). By adding ozonized olive oil at %10-50 ratio, the consistency and the paramagnetic effect will be enhanced and rectal use can be achieved and upper gastrointestinal system can be viewed.

The invention in subject; is gastrointestinal purposed with its' elementary chemical characteristics and can also be considered to be developed as an intravenous MR contrast agent. The animal tests conducted have shown that the invention is also helpful in viewing the urinary system (MR Urography). By adding sucralfate or adhesion enhancing substances through nanotechnologic methods, it's also possible in theory to be used as a highly mucoadhesive product in stomach and intestines.

In a more preferred embodiment, the contrast agent composition is obtained by the following steps of processes; washing 500 gr of Beta Vulgaris (BV) plant with water (10), peeling and slicing it (20), pulping it (30), boiling it (40), cooling it (50), draining it (60), separating the liquid part of the material (70), adding garlic, 4-5 branches of saffron and cilantro on 150 ml liquid to enhance the flavor (80), obtaining Betanin and Vulgaxanthin extract from the particles of Beta Vulgaris (BV) through ethanoic extraction method (90) and adding betanin (3/4) and vulgaxanthin (1/4) onto the 150 ml liquid (80) in a 30 ml extract form (100). (FIG. 1, FIG. 2). If the purpose is to use it rectally and view the upper gastrointestinal system, ozonized olive oil can be added onto it at a ratio of %10-50. (FIG. 1, FIG. 2). The invention in subject; can be used as a gastrointestinal and intravenous MR contrast agent with its' elementary chemical characteristics. Its to be used as a highly mucoadhesive product in stomach and intestines by adding sucralfate or adhesion enhancing substances through nanotechnologic methods. By adding ozonized olive oil at %10-50 ratio, the consistency and the paramagnetic effect is enhanced and rectal use can be achieved or upper gastrointestinal system can be viewed.

Its known that Beta Vulgaris extract contains high amounts of nitric oxide. The substance obtained are also examined for elementary and food analysis.

The present invention may be further summarized by reference to the following clauses #1-#4:
#1. This invention is the phytochemical agent containing betalain used in magnetic resonance imaging (MRI) and that enhances the T1 and T2 contrast quality and its' characteristic is and its' characteristic is being obtained by the following steps of processes;
   washing 500 gr of Beta Vulgaris (BV) plant with water (10),
   peeling and slicing it (20),
   pulping it (30),
   boiling it (40),
   cooling it (50),
   draining it (60),
   separating the liquid part of the material (70),
   adding garlic, 4-5 branches of saffron and cilantro on 150 ml liquid to enhance the flavor (80),
   obtaining Betanin and Vulgaxanthin extract from the particles of Beta Vulgaris (BV) through ethanoic extraction method (90) and
   adding betanin (3/4) and vulgaxanthin (1/4) onto the 150 ml liquid (80) in a 30 ml extract form (100).
#2. What's mentioned in Claim 1 is the phytochemical agent containing betalain used in magnetic resonance imaging (MRI) and that enhances the T1 and T2 contrast quality and its' characteristic is and its' characteristic is; by adding ozonized olive oil at %10-50 ratio, the consistency and the paramagnetic effect being enhanced and rectal use being achieved or upper gastrointestinal system being viewed.
#3. What's mentioned in Claim 1 is the phytochemical agent containing betalain used in magnetic resonance imaging (MRI) and that enhances the T1 and T2 contrast quality and its' characteristic is and its' characteristic is; being used as a gastrointestinal and intravenous MR contrast agent with its' elementary and food chemical-physical characteristics.
#4. What's mentioned in Claim 1 is the phytochemical agent containing betalain used in magnetic resonance imaging (MRI) and that enhances the T1 and T2 contrast quality and its' characteristic is and its' characteristic is; being used as a highly mucoadhesive product in stomach and intestines by adding sucralfate or adhesion enhancing substances through nanotechnologic methods.

To further illustrate the invention, the following examples are provided. These examples are provided with no intend to limit the scope of the invention.

### I. Preparation of Contrast Agent Compositions According to the Present Invention

I.1. Preparation of a contrast agent composition according to the present invention (CA1) 500 g of Beta vulgaris (BV) plant originating from Aydin region (Turkey) was washed with water (10), peeled and sliced (20), pulped (30), mixed with 500 mL water and boiled for 2 hours (40). Following cooling (50) and draining (60), 150 mL of liquid extract (CA1) was obtained (70). The liquid extract (CA1) can be used directly as a contrast agent for magnetic resonance imaging. The composition CA1 is particularly suitable for oral administration and imaging of upper gastrointestinal tract. For oral administration, garlic and/or branches of saffron and/or branches of cilantro may be added to the liquid extract to enhance the flavor (80). The preparation method is summarized by FIG. 1.

I.2. Preparation of a contrast agent composition according to the present invention (CA2, CA6) 27.75 g (30 ml) of commercially available beetroot red pigment (CAS Nr 7659-95-2, Turkish Food additive number E162; Supplier: Smart Kimya Tic. ve Dan. Ltd. Şti. Ege Sanayi Sitesi Balatçik Mah. 8901/3 Sok. No: 3/3AO Çiǧli/Izmir, https://magaza.hammaddeler.com/kategori/pancar-koku-kirmizisi-1) was dissolved in 150 ml of the liquid extract CA1 (80) obtained as described at example 1 to provide a solution (CA2) that can be used directly as a contrast agent for magnetic resonance imaging with higher contrast compared to (CA1). The commercially available beetroot red pigment contains 75 wt-% of betanin and 25 wt-% of vulgaxanthin. The composition (CA2) is particularly suitable for oral administration. The composition has a pH of 3.87, and a concentration of betanin of 924.5 mg/100 g as determined by HPLC. The HPLC analysis was performed on a Shimadzu Prominence LC20A and Shimadzu SPD-20A HPLC instrument. The samples were passed through a 0.45 μm PVDF filter and transferred into vials to be injected into the HPLC (Shimadzu Prominence LC20A). In the study using UV detector (Shimadzu SPD-20A) and reverse phase C18 column (250 mm×4.60 mm×5 μm), isocratic mobile phase was 0.5% trifluoroacetic acid solution and acetonitrile 90:10 (v:v). Analysis at 540 nm, 1 mL/min. flow rate and column temperature of 20° C. Identification and quantification were performed using the betanin standard.

The preparation method is summarized in FIG. 2.

0.5 ml of CA2 were mixed with 1.5 ml water to provide contrast agent composition (CA6) according to the present invention having a concentration of betanin of approximately 231 mg/100 g.

### I.3. Preparation of a Contrast Agent Composition According to the Present Invention (CA3)

50 mL of the composition (CA2) obtained as described at example 2 were mixed with 50 mL of olive oil. The obtained mixture was ozonized for three hours to provide an emulsion (CA3) that can be used directly as a contrast agent for magnetic for resonance imaging. The composition (CA3) is particularly suitable for rectal administration.

### I.4. Preparation of a Contrast Agent According to the Present Invention (CA4, CA5)

Solution CA4 and solution CA5 were prepared by dissolving 500 mg (CA4) and 250 mg (CA5) of commercially available betanin (red beet extract diluted with dextrin, CAS Nr.: 7659.95-2; supplier: Sigma Aldrich Chemie) in 1 mL of saline 1 M. Both solutions can be used as a contrast agent for magnetic resonance imaging. The composition CA4 and CA5 are particularly useful for oral, intraperitoneal and intravenous administration. The compositions may be mixed with olive oil in a volume ratio from about 9:1 to about 1:1 and ozonized to provide emulsions particularly suitable for rectal administration.

### I.5. Preparation of a Gadolinium Chelate Solution for Comparative Purposes (C1)

1 mL of Gadovist® (MRI contrast agent containing 1 mmol gadolinium chelate/mL; supplier: Bayer) was diluted with 100 mL water to provide a solution (C1) to be used for comparative purposes.

### II. In Vitro Evaluation of the MRI Contrasting Properties of the Compositions According to the Present Invention

The contrast agent compositions CA1, CA2, CA5 and CA6 were tested on a 3T MR scanner in comparison with gadolinium chelate solution C1, oil (extra virgin olive oil) and water. T1 relaxation graphic was generated based on multiple TR and TE values. T1 and T2 were measured by following the established procedures described in Magnetic Resonance in Medicine 46:1099-1106 (2001). Briefly, the samples (CA1, CA2, CA5, CA6, C1, water and oil) were poured directly in conic and cylindrically shaped plastic containers. The holder was inserted into head-only volume coil and imaged on coronal and axial plane with a 3T Scanner (GE Pioneer, 3T). Standard spin echo sequence executed for different TR values (75, 150, 300, 600, 1000, 2000 and 4000 ms) while TE=10 ms, and various TE values (10, 40, 80 and 120 ms) with TR=4000 ms. FIG. 9 is one of the acquired slices where each disc represent one of the samples CA5, CA6, C1 and water. The contrast variation from sample to sample indicates different T1 signal intensity.

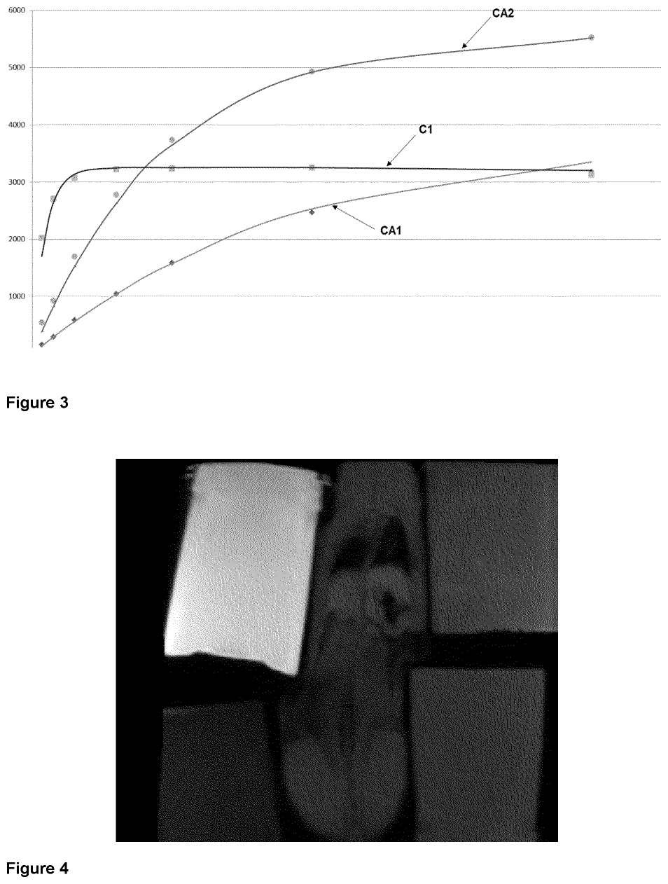

Regions of interest were placed on each sample and average reading of the intensity was recorded in a table. FIG. 3 shows a comparative schematic view of the T1 relaxation curves measured for the contrast agent compositions CA1 and CA2 according to the present invention and the comparative gadolinium chelate composition (C1) (x axis: time of repetition (TR)/ms; y axis: T1 average image signal intensity over selected region of interest. The contrast compositions CA1 and CA2 according to the present invention exhibit a spin echo T1 comparable with the currently used gadolinium chelate composition C1. Hence, contrast compositions CA1 and CA2 are suitable to be used as a contrast agent for in vivo magnetic resonance imaging.

### III. In Vivo Evaluation of the MRI Contrasting Properties of the Compositions According to the Present Invention

### III. 1 General Procedure for Measuring the MRI Signal on Rats

Rats were imaged under ketamine (50 mg/kg) and Xylazine (5 mg/kg) anesthesia using flexible wrist coil with 3 T MRI scanner T1-weighted images were acquired in coronal plane with parameters (TR=75, TE=10, Slice thickness=10, FOV=24).

### III.2 General Procedure for Magnetic Resonance Imaging on Humans

The abdominal study were performed with a 1.5 Tesla scanner (Philips Achieva, Eindhoven, The Netherlands) before and after administration of freshly pressed beetroot juice. Breathold spoiled gradient echo (GRE) axial and coronal in-phase gradient echo (GRE) (TE: 4.2 ms, TR: 8.5, FA: 10, Slice thickness: 10 mm), in phase and out of phase T1 images were obtained. Following T1 images, axial and coronal T2 weighted turbo spin echo images were generated using TE: 80 ms, TR: 556, FA: 90, Slice thickness: 7 mm), for gastric imaging.

### III.3 MRI on Rats

Experiments were started after the approval of the local ethics committee for animal experiments (Approval number: 64583101/2021/067). Animal study was carried out using 10 Wistar albino rats weighing 250-300 g for 12 weeks (Control group, n=5, MRI group n=5). During the experimental study, the rats were housed at 22±2° C. temperature and 12/12, light/dark conditions. Feed and water were administered ad libitum.

### III.3.1 Oral Administration of the Contrast Agent Composition According to the Present Invention

Prior to administration, the rats were fasted for 12 hours before measuring the MRI signal. Then, the contrast composition according to the present invention (CA2) and the comparative gadolinium chelate solution (C1) was applied with oral gavage (Harward apparatus, 22G) at a dose of 3 ml. Ketamine (Ketasol %10 Interhas, Ankara/Turkey) 50 mg/kg and xylazine (Xylazinobio %2, Bioveta/Czechia) 5 mg/kg were applied for sedation just before the MRI imaging. Rats were kept stable and motionless with the help of a platform. One rat was imaged on MRI for 1 hour at 10-minute intervals and also for late phase to see the parenchymal organ enhancement. For late phase measurement, the MRI was performed three hours after administration. The rest of the rats were imaged for 10 minutes for the total exam. The captured images were saved for analysis and evaluation.

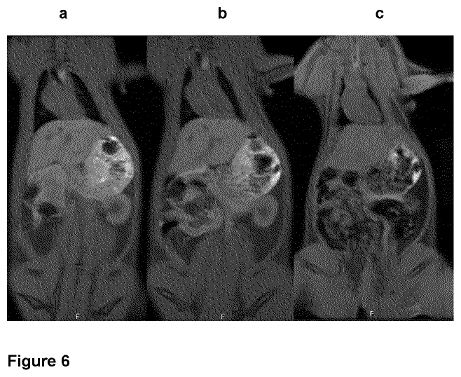

The MRI results obtained with the contrast composition CA2 are shown in FIG. 6. As can be seen from FIG. 6, the contrast agent composition CA2 shows bright MRI contrast in stomach following oral administration, enables delineation of the stomach lumen and is particularly suitable for gastro-esophageal imaging. The black portions noticed in the stomach correspond to sawdust.

The MRI results obtained with the contrast composition CA2 are shown in FIG. 7 A): early phase (acquisition after first minute post administration) spin echo T1 weighted image (image a), early phase (acquisition after first minute post administration) fat saturated T2 weighted imaged (image b), and early phase (acquisition after first minute post administration) sequential gradient echo T1 weighted images (images c and d), and late phase (acquisition after three hours post administration) spin echo T1 weighted image (image e). As can be seen from FIG. 7 A), contrast agent composition CA2 enables delineation of the small intestine at early phase and of the liver and kidneys at late phase post oral administration and is particularly useful for upper gastrointestinal imaging, and parenchymal imaging.

The MRI results obtained with the comparative gadolinium chelate solution C1 are shown in FIG. 7 B): early phase (acquisition after first minute post administration) spin echo T1 weighted images (image a and b), and early phase (acquisition after first minute post administration) sequential gradient echo T1 weighted images (image c).

Comparison of the results depicted by FIGS. 7A and 7B demonstrates that the contrast composition CA2 according to the present invention shows better distention and delineation of the lumen of small bowel especially at sequential gradient echo T1 weighted imaging than the currently used gadolinium chelate composition (C1). This is particularly advantageous because the measurement of a gradient echo on a LAVA ASPIR MRI sequence T1 weighted image is more expedient than the one of a spin echo T1 weighted image.

### III.3.2 Rectal Administration of the Contrast Agent Composition According to the Present Invention

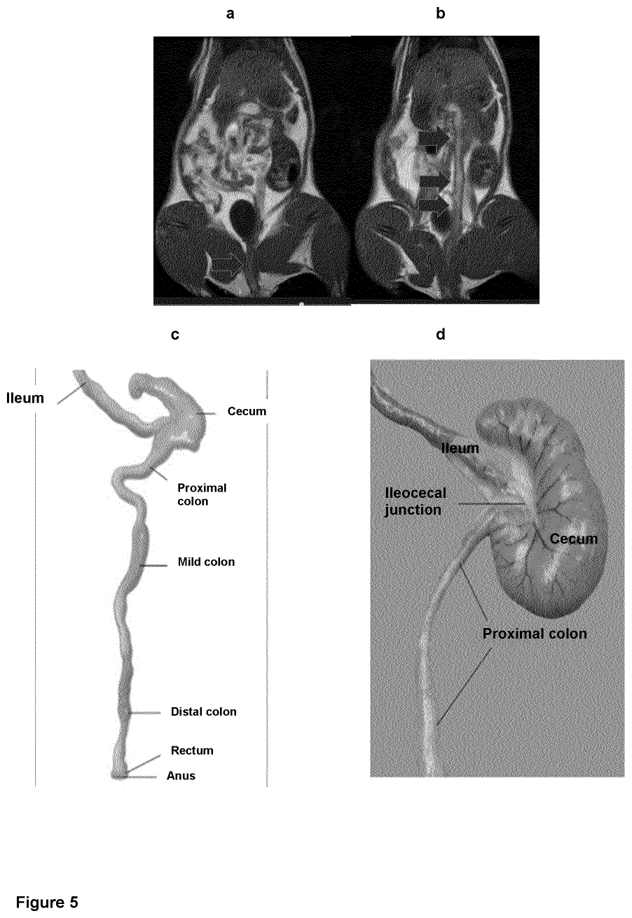

Prior to administration, the rats were fasted for 12 hours before measuring the MRI signal. Then, the contrast composition according to the present invention (CA3—FIG. 5) was applied rectally at a dose of 1 ml. Ketamine (Ketasol %10 Interhas, Ankara/Turkey) 50 mg/kg and xylazine (Xylazinobio %2, Bioveta/Czechia) 5 mg/kg were applied for sedation just before the MRI imaging. Rats were kept stable and motionless with the help of a platform. MRI was performed for 1 hour at 10-minute intervals from each rat. The captured images were saved for analysis and evaluation.

The results obtained with the contrast agent composition CA3 are depicted in FIG. 5. As shown by images a and b of FIG. 5, the contrast agent composition CA3 shows bright MRI contrast in intestine following rectal administration, enables delineation of the bowel lumen and is particularly suitable for lower gastrointestinal imaging.

### III.3.3 Intravenous Administration of the Contrast Agent Composition According to the Present Invention

1.5 mL of the contrast agent composition CA4 was manually administered via tail veins. Ketamine (Ketasol %10 Interhas, Ankara/Turkey) 50 mg/kg and xylazine (Xylazinobio %2, Bioveta/Czechia) 5 mg/kg were applied for sedation just before the MRI imaging. Rats were kept stable and motionless with the help of a platform. MRI was performed for 1 hour at 10-minute intervals from each acquisition. The captured images were saved for analysis and evaluation

The results obtained with the contrast agent composition CA4 are depicted in FIG. 10: early (acquisition after first minute post manual injection) rat substracted angiographic imaging (image a), late phase (acquisition after second minute post manual injection) rat substracted angiographic imaging (image b) and late phase (acquisition after second minute post manual injection) rat substracted abdominal imaging (image c) imaging. FIG. 10 shows venous phase and parenchymal phase (liver) and demonstrates that the composition CA4 is particularly useful for vessels and parenchymal imaging following intravenous administration.

### III.4 Acute Feeding Study

In order to investigate the possible acute toxicity effects of the contrast agent composition CA2, feeding trials were carried out for 2 and 4 days. For this purpose, 15 rats were divided into 3 groups as control (n=5), 48-hour group (n=5) and 96-hour group (n=5). While the control group was given isotonic saline, 3 ml of the contrast agent composition CA2 was administered orally to the feeding groups for 2 and 4 successive days. Blood samples were taken intracardiacly. From blood serum samples, ALT (Alanin Aminotransferase), and AST (Aspartate Aminotransferase), Urea and Creatine values, TAS (Total Antioxidant Status) and TOS (Total Oxidant Status) levels were evaluated (Clin Biochem, 38 (12): 1103-1111, 2005) At the end of the feeding trial, the rats were euthanized by cervical dislocation under anesthesia (Ketamin and Ksilazin).

### Measurement of Total Oxidant Status (TOS)

TOS of serum were determined using a novel automated measurement method described in Clin Biochem, 38 (12): 1103-1111, 2005. Oxidants present in the sample oxidize the ferrous ion-o-dianisidine complex to ferric ion. The oxidation reaction is enhanced by glycerol molecules, which are abundantly present in the reaction medium. The ferric ion makes a colored complex with xylenol orange in an acidic medium. The color intensity, which can be measured spectrophotometrically, is related to the total amount of oxidant molecules present in the sample. The assay is calibrated with hydrogen peroxide and the results are expressed in terms of micromolar H₂O₂ equivalent per liter (μmol H₂O₂ equiv./L) (1).

### Measurement of Total Antioxidant Status (TAS)

TAS of serum were determined using an automated measurement method described in Clin Biochem, 37 (2): 112119, 2004. The method is based on the bleaching of the characteristic color of a more stable 2,2′-azino-bis (3-ethylbenz-thiazoline-6-sulfonic acid) (ABTS) radical cation by antioxidants. The results were expressed in mmol Trolox equivalents/L.

The results of the blood analysis are summarized in Table 1. As demonstrated by the TAS values, the contrast agent composition CA2 presents an antioxidant activity. No renal or liver toxicity could be observed following administration of the contrast agent composition CA2.

TABLE 1
Blood biochemical values of rats fed with oral phytochemical
extract for 2 and 4 days

| | TAS | Urea | Creatine | ALT | AST |
|---|---|---|---|---|---|
| Groups | (mmol/L) | (mg/dl) | (mg/dl) | (U/L) | (U/L) |
| Control | 1.76 ± 0.04 | 44.5 ± 1.70 | 0.57 ± 0.02 | 44.5 ± 7.38 | 80.75 ± 3.86 |
| 2 days | 2.55 ± 0.59 | 30.5 ± 4.11 | 0.53 ± 0.03 | 39.5 ± 14.93 | 76.75 ± 6.02 |
| feeding | | | | | |
| 4 days | 2.53 ± 0.67 | 31.25 ± 2.62 | 0.53 ± 0.03 | 51.0 ± 7.08 | 79.25 ± 7.18 |
| feeding | | | | | |

Liver and kidney tissues were taken, put into 10% formol solution and histopathologically evaluated in terms of possible effects.

### Histological Procedure

Liver and kidney tissues were taken, put into 10% formal solution and histopathologically evaluated in terms of possible effects. No pathology has been observed on the studied tissues.

Stomach and bowel tissues were placed in a 10% formaline solution for 24 hours before processing and embedding in paraffin wax. Paraffin-embedded tissues were cut at 4 μm, prepared and dyed with hematoxylin-eosin (HE) and examined under light microscopy (Olympus BX50; Olympus Corp., Tokyo, Japan). Light two microscopic analysis of the tissue specimens were performed by blinded observation. FIG. 11 A) shows a microscopic image of a histhological sample from the experimented rat's bowel following oral administration for four consecutive days of the contrast composition CA2 according to the present invention. FIG. 11 B) shows a microscopic image of a histhological sample from the experimented rat's stomach following oral administration for four consecutive days of the contrast composition CA2 according to the present invention. The results displayed by FIG. 11 demonstrate that there is no toxic effect associated with the oral administration of a dosis of 1 mL of the contrast agent composition CA2 according to the present invention for four consecutive days.

### III.5 Magnetic Resonance Imaging on Humans

The inventor starved for 12 hours prior to drinking 400 ml of freshly pressed beetroot juice. The MRI results are displayed by FIG. 8: MRI images the abdomen of the inventor prior (FIG. 8 a: left image: axial gradient echo T1 weighted image; right image: coronal gradient echo T1 weighted imaged) and after oral administration of the freshly pressed beetroot juice (FIG. 8 b: left image: axial gradient echo T1 weighted image (out of phase); right image: coronal gradient echo T2 weighted imaged). As seen in FIG. 8, the freshly pressed beetroot juice enables better delineation of human stomach lumen and wall following oral administration and is particularly useful for gastric imaging.

### III.6 Molecular Analyses on Human Normal Fibroblast Cell Culture

### CA2 was Tested in Human Healthy Fibroblast Cell Culture and Showed No Antiproliferative Effect and No Effect on Rate of Cell Viability.

Human, neonatal, healthy primary dermal fibroblast (HFB, ATCC® PCS-201-010™) were maintained in Dulbecco's modified Eagle medium supplemented with 10% heat-inactivated fetal bovine serum (FBS), 50 U/ml penicillin, and 50 μg/ml streptomycin. The cells were cultured in a 95% humidified atmosphere containing 5% CO2 at 37° C. These adhesive cells were passaged every three days by trypsinization and washing with Ca and Mg-free phosphate-buffered saline (PBS). Then, the cells were fed with the same media. They were seeded on a 96-well plate 24 hours before starting the experiments. 1×10⁴ cells were plated for each well. After 24 hours, cells were incubated with different concentrations (0-60 μM) of contrast agent composition CA2 for 24 hours. Then, an XTT assay was performed.

### Cell Proliferation Assay:

Cell proliferation was studied according to the manufacturer's instructions with an XTT-based kit (Biological Industries, cat. No: 20-300-1000) and measured spectrophotometrically at 450/670 nm with the TECAN Sunrise instrument three times at 24 h after the CA2 administration.

FIG. 12 shows the cells proliferation levels in human healthy fibroblast (HFB) cells after administration of a contrast agent composition according to the present invention (CA2) at a concentration of between 0-60 μM (x-axis: applied concentrations of CA2, y-axis: absorbance values measured in the spectrophotometry device (TECAN Sunrise)): the effect of CA2 administered between 0-60 μM in human healthy fibroblast (HFB) cells on the viability of cells after 24 hours of incubation. No statistically significant difference was observed.

1.-11. (canceled)

12. A method of acquiring a contrast enhanced magnetic resonance image of a subject, said method comprising
providing a composition comprising:
i) water, and
ii) one or more chemically distinct betalains:
administering to said subject said composition; and
acquiring said magnetic resonance image of said subject.

13. The method according to claim 12, wherein the composition is administered orally or rectally.

14. The method according to claim 12, wherein the esophagus, the gastro-intestinal tract, the liver, and/or the kidney of said subject is visible in said magnetic resonance image.

15. The method according to claim 12, wherein the composition is administered parenterally, preferably intravenously, or intraperitoneally.

16. The method according to claim 15, wherein the venous system and/or the arterial system is visible in said magnetic resonance image.

17. The method according to claim 12, wherein the one or more chemically distinct betalains are selected from compounds of general formula (I) 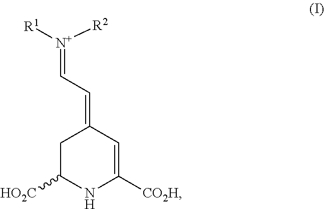
wherein in general formula (I)
either
R¹ represents —H, and R² is selected from 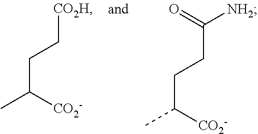
or
R¹ and R² together with the nitrogen to which they are attached form a residue of general formula (II) or (III) 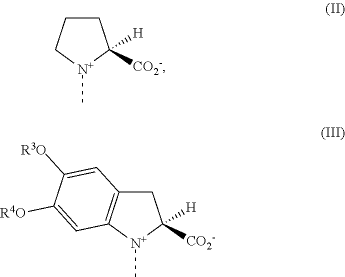
wherein in general formula (III),
R³ and R⁴ are independently of each other selected from
—H, and 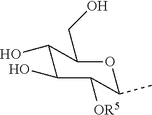
with the proviso that at least one of R³ and R⁴ represents —H, and
R⁵ is selected from —H and 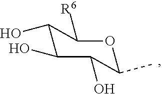
with R⁶ being selected from —CH₂OH and CO₂H, pharmaceutically acceptable salts thereof, and tautomers thereof.

18. The method according to claim 12, wherein the one or more chemically distinct betalains comprise a compound of general formula (IV) 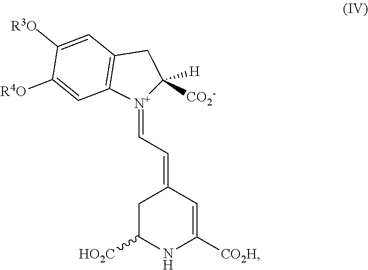
wherein R³ and R⁴ have the meanings defined in claim 2, a pharmaceutically acceptable salt thereof and/or a tautomer thereof, and
a compound of general formula (V) 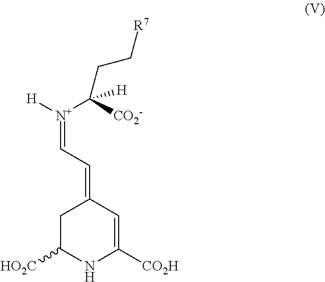
wherein in general formula (V) R⁷ is selected from —CO₂H and —CONH₂, a pharmaceutically acceptable salt thereof and/or a tautomer thereof.

19. The method according to claim 12, wherein the one or more chemically distinct betalains are selected from compounds of general formula (IV) 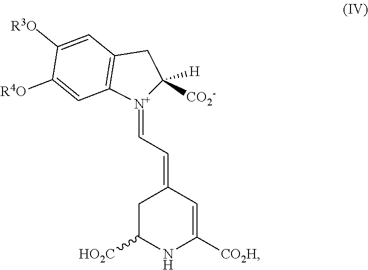
with R³ and R⁴ having the meanings as defined in claim 2, pharmaceutically acceptable salts thereof, and tautomers thereof.

20. The method according to claim 19, wherein the compound of general formula (IV) is present in the composition in an amount from about 2 g/kg to about 10 g/kg as determined by High Pressure Liquid Chromatography.

21. The method according to claim 12, wherein the composition is a Beta vulgaris juice.

22. The method according to claim 12, wherein the composition is obtained by the process containing the steps:
a) peeling and slicing a Beta vulgaris plant;
b) pulping the Beta vulgaris plant to provide a pulp;
c) mixing the pulp obtained at step b) with water;
d) boiling the mixture obtained at step c);
e) following cooling, draining the mixture to separate a liquid; and optionally
f) adding an aqueous solution containing a betanin and/or a vulgaxanthin, to the liquid obtained at step e).

23. The method according to claim 12, wherein the composition further contains up to 50 vol-% olive oil, preferably from 10 to 50 vol-% olive oil.

24. The method according to claim 12, wherein the composition is ozonized.

25. The method according to claim 18, wherein the compound of general formula (IV) is present in the composition in an amount from about 2 g/kg to about 10 g/kg as determined by High Pressure Liquid Chromatography.

26. A process for manufacturing the composition recited in claim 12, wherein said process contains the following steps:
a) peeling and slicing a Beta vulgaris plant;
b) pulping the Beta vulgaris plant to provide a pulp;
c) mixing the pulp obtained at step b) with water;
d) boiling the mixture obtained at step c);
e) following cooling, draining the mixture to separate a liquid; and
f) adding an aqueous solution containing a betanin and/or a vulgaxanthin to the liquid obtained at step e).

27. A contrast agent composition for magnetic resonance imaging obtained by the process according to claim 26.
